(19) United States
(12) Patent Application Publication
(10) Pub. No.: US 2002/0095089 A1
(43) Pub. Date: Jul. 18, 2002

(54) Amusement system based on an optical instrumentation method for the living body

(75) Inventors: Tsuyoshi Yamamoto, Hatoyama (JP); Atsushi Maki, Hachioji (JP); Michiharu Nakamura, Tokyo (JP); Hideaki Koizumi, Tokyo (JP)

Correspondence Address: Stanley P. Fisher, Reed Smith Hazel & Thomas LLP, Suite 1400, 3110 Fairview Park Drive, Falls Church, VA, 22042-4503, US

(73) Assignee: Hitachi, Ltd.

(21) Appl. No.: 09/925,325

(22) Filed: Aug. 10, 2001

(30) Foreign Application Priority Data
Dec. 7, 2000 (JP) 2000-373292

(51) Int. Cl. A61B 5/00

(52) U.S. Cl. 600/476; 128/897

(57) ABSTRACT

An amusement system is provided wherein a variation of light intensity transmitted through a living body which reflects a metabolite concentration or concentration variation in the living body reflecting a mental state or brain activity, is measured, and the measurement result is reflected in an object displayed on a screen. By bringing a light illumination device and light detector in contact with the skin of a subject, the intensity of the light propagated in the subject is detected, and the detection result is transmitted to an electronic calculator. The position, shape or color of the object displayed on the screen is made to vary according to the variation of this detected intensity. Thus, human thought can be measured and the state of the object on the screen controlled without using an existing input device such as a mouse, joystick or handle.

### BACKGROUND OF THE INVENTION

The present invention relates to an amusement system, and in particular an amusement system using an optical instrumentation method for the living body which measures change of light intensity penetrating the living body, reflecting a metabolite concentration or concentration change in the living body indicative of a mental state and brain activity.

A living body input device and biological control device using an optical instrumentation method for the living body which controls a computer, a game, an environmental control device, a learning degree determining device, a vehicle warning device, medical diagnosis and warning device, a lie detector, a declaration of intention device or a data transmission unit, etc. which function by measuring a localized brain function and inputting it into an external device, are proposed in Japanese Patent Application Laid-Open No. H9-149894.

Hereafter, this will be described using FIG. 19.

In order to irradiate a subject with light, a light source (19-1) represented by a semi-conductor laser, a light emitting diode or a lamp, and a light waveguide (19-2) represented by an optical fiber for illumination (referred to collectively as optical illuminator), are used.

Although it is optimal to use light having a wavelength of about 800 nm to which living tissue is highly permeable as the wavelength of the light used for this measurement, the method is not limited to this wavelength band.

The two ends of the light waveguide respectively touch the light source (19-1) and the skin of the subject (19-3) The light irradiated to the living body is strongly scattered by the living tissue. However, part of the scattered light passes through the cerebral cortex in which high order brain functions such as movement, feeling and language are concentrated, and again arrives at the scalp about 3 mm (in the case of an adult) away from the light illumination position.

A light detector is provided in order to detect the intensity of the light propagated in the living body at this position. This light detector comprises a light waveguide (19-4) such as an optical fiber, and a photoelectric element (19-5) such as a photodiode or photomultiplier with which one end of the waveguide is in contact. The optical signal is changed into an electrical signal using this light detector, and this electrical signal is processed by a computer (19-6).

Here, it is assumed that the brain is made to work by moving the body (a hand, leg or fingers, etc.), thinking about something, or concentrating on something. When the brain works, in order to supply oxygen and glucose to the cerebrally active part, the blood volume in the cerebral cortex changes (increases or decreases) secondarily. If near infrared light (wavelength of about 800 nm) is used for the measurement, as the hemoglobin in blood (oxidized hemoglobin or reduced hemoglobin) absorbs this light, the light amount reaching the optical fiber for detection will decrease if the hemoglobin amount increases in connection with brain activity. For this reason, a change in the intensity of the detected light reflects cerebral activity. Thus, an input device has been realized wherein, by measuring the intensity variation of this light and controlling a computer using the measurement result, human thinking reflecting a mental state or brain activity is measured to control a computer.

In this invention, the following two problems are solved.

Firstly, an amusement system using the above-mentioned living body optician instrumentation device is provided. In general, an amusement system comprises an input device such as a mouse, a joystick, a handle or a touch panel, and a display device which displays the input result to the player such as a Braun tube display, liquid crystal display or light emitting diode array. A common feature of these existing input devices is that a hand or foot is moved based on the instructions from the brain, and the instructions from the brain are input into the computer using the hands or feet. Various amusement systems have already been realized by varying a “state” such as the position, form or size of an object displayed on the display device according to this input.

In the living body input device disclosed in Japanese Patent Application Laid-Open No. H9-149894, although it is possible to input human thought into a computer without using the hands or feet, the actual means of implementing an amusement system using this living body input device is not mentioned. If such an amusement system were realized, it would provide a new amusement system not only for healthy persons, but also for persons who have difficulty in moving their hands or legs, and various persons could therefore enjoy themselves using the same amusement system.

### SUMMARY OF THE INVENTION

It is therefore a first object of the invention to directly input commands from the brain and brain activity into a computer using an optical brain function measuring method, and to provide an amusement system based on the input result.

Specifically, in the first instance, a human brain function which is activated when a man thinks about something, concentrates on something or attempts to move a hand or leg is measured using light. In the second instance, an amusement system is provided which makes it possible to reflect the result of measuring this human brain activity in an object displayed on a computer screen.

It is a second object of this invention to provide a game in the aforesaid amusement system where the player does not experience fatigue due to over-concentration. In the above amusement system, a computer is controlled using human brain activity. As human brain activity is controlled using the brain, there is a risk of inducing fatigue. Also, if the game is interesting to the player, there is a risk that the player will forget the passing time, become over-involved in the game and therefore experience fatigue. Hence, a game is provided which does not cause fatigue.

The human brain may be divided into measurement regions with different cellular structures as shown on the map of Broadman, and these regions have different functions. For example, when the brain is viewed from the side, the region concerned with spontaneous movements (hand, leg or fingers) is the tip of the head, the region concerned with feeling and vision is the rear of the head, and the region concerned with language is a predetermined part of the left half.

According to this invention, as information is extracted with high precision from positions specified in this way, an optical instrumentation method for the living body having a high spatial resolution is used (in the measurement of brain waves, the dielectric constant in the living body is uneven, so the position where a signal originates is ill-defined and spatial resolution is low. Further, as the myoelectric potential is largely reflected in the signal relative to motion of the body of the subject, there is also a disadvantage in that the subject has to be restrained.

This optical instrumentation method for the living body comprises at least one light illumination device disposed on the skin of one or more subjects, at least one light detector disposed on the skin of the subject for condensing light transmitted in the skin of the subject due to illumination of the skin of the subject by the light illumination device, and measuring the transmission intensity of the condensed light, and a computing part which calculates the concentration variation of a metabolite in the living body measured using the light illumination device and light detector.

It is a feature of the amusement system based on this invention that the computing part of the optical instrumentation method for the living body is connected to a display unit comprising a screen. At least one of a certain object is displayed on the screen of this display unit. It is a characteristic feature that a “state” such as the position, form or size of the object varies according to the intensity variation of the signal light transmitted through the living body. Consequently, the concentration change of the metabolite in the living body due to brain activity can be visualized. As a result, the subject can vary the “state” such as the position, form or size of the object displayed on the screen by activating his/her own brain, thereby implementing the amusement system which is the object of this invention. The computing part and display screen may be formed in one piece or independently.

This invention therefore provides an amusement system using an optical instrumentation method for the living body comprising at least one light illumination device for irradiating the subject with light, at least one light detector which condenses the transmitted light irradiated by the light illumination device and propagated through the living body, a display unit provided with a screen which displays at least one object, and a computing part which controls the display unit based on a measurement signal related to the intensity of the transmitted light measured by the light detector, wherein a state comprising at least one of the objects displayed on the screen varies according to the intensity variation of the signal measured by the one or more light detectors.

This invention further provides an amusement system using a living body optical instrumentation method comprising at least one light illumination device for irradiating the subject with light, at least one light detector which condenses the transmitted light irradiated by the light illumination device and propagated through the living body, a computing part provided with a display screen which displays at least one object, and a computing part which controls the display unit based on a measurement signal related to the intensity of the transmitted light measured by the light detector, wherein a state comprising at least one of the objects displayed on the screen varies according to the intensity variation of the signal measured by the one or more light detectors.

This invention further provides an amusement system using an optical instrumentation method for the living body comprising at least one light illumination device which irradiates plural living bodies with light via waveguides, at least one light detector which condenses the transmitted light irradiated by the light illumination device and propagated through the living body, a display unit provided with a screen which displays at least one object, and a computing part which controls the display unit based on a measurement signal related to the intensity of the transmitted light measured by the light detector, wherein a state comprising at least one of the objects displayed on the screen various according to the intensity variation of the measured signal measured by one or more light detectors.

This invention further provides an amusement system using an optical instrumentation method for the living body wherein the light illumination device, light detector, display unit and computing unit are built into the same information terminal, and parts of the light illumination device and light detector are connected to a probe of the information terminal.

This invention further provides an amusement system using an optical instrumentation method for the living body wherein the computing part has a function for storing the cumulative time and a reference illumination period during which light is irradiated by the light illumination device, and a speaker which emits a sound, and when the cumulative time during which light is irradiated by the light illumination device exceeds the reference illumination period, a command is issued to modify the arrangement on the display screen or a command is issued to modify the sound emitted by the speaker.

This invention further provides an amusement system using an optical instrumentation method for the living body wherein the computing part issues a command to modify the arrangement on the display screen or modify the sound emitted by the speaker when the intensity of the transmitted light exceeds a predetermined threshold value even if the cumulative time during which light is irradiated by the light illumination device is within the reference illumination period.

### BRIEF DESCRIPTION OF THE DRAWINGS

FIG. 1 is a diagram showing an amusement system (1) based on an optical instrumentation method for the living body.

FIG. 2A is a drawing showing an image, before brain activity, of brain structure, light propagation characteristics in the living body and concentration increase of a metabolite in the living body due to brain activity.

FIG. 2B is a drawing showing an image, after brain activity, of brain structure, light propagation characteristics in the living body and concentration increase of a metabolite in the living body due to brain activity.

FIG. 3A is a diagram describing an intensity variation of light propagated in the living body before and after a brain activity period

FIG. 3B is a diagram describing an example of a method of displaying an object on a screen reflecting this intensity variation.

FIG. 4 is a diagram showing an amusement system (2) based on the optical instrumentation method for the living body—method of measuring plural points on the subject.

FIG. 5 is a diagram describing an example of a method of displaying an object on a screen using the measurement results based on the optical instrumentation method for the living body shown in FIG. 4.

FIG. 6 is a diagram showing an amusement system (3) based on the optical instrumentation method for the living body—method of simultaneously measuring plural subjects (1).

FIG. 7 is a diagram showing an amusement system (4) based on the optical instrumentation method for the living body—method of simultaneously measuring plural subjects (2).

FIG. 8 is a diagram showing a measurement sequence in implementing the amusement system shown in FIG. 7.

FIG. 9 is a diagram describing an example of a method of displaying an object on a screen using the measurement results based on the optical instrumentation method for the living body shown in FIGS. 6 and 7.

FIG. 10 is a diagram showing an amusement system (5) based on the optical instrumentation method for the living body—method of simultaneously measuring plural points on plural subjects.

FIG. 11 is a diagram describing an example of a method of displaying an object on a screen using the measurement results based on the optical instrumentation method for the living body shown in FIG. 10.

FIG. 12 is a diagram describing an example of an instrumentation method for the living body using an information terminal, and a method of displaying an object on a screen using the measurement results.

FIG. 13 is a diagram showing the construction of an instrumentation device for the living body connected to an information terminal.

FIG. 14 is a diagram describing an example wherein a blood volume change due to brain activity in a subject is shown by varying the size of an object displayed on a screen.

FIG. 15 is a diagram describing an example wherein a blood volume change due to brain activity in a subject is shown by varying the color of an object displayed on a screen.

FIG. 16 is a diagram relating to an example (limiter) of an amusement system which does not easily cause a subject to experience fatigue.

FIG. 17A is a diagram showing a trigger occurrence interval for inspecting transmitted light intensity, in an example of an algorithm for determining the end of a measurement by a limiter (or a game).

FIG. 17B is a diagram showing a time dependency of transmitted light intensity, in an example of an algorithm for determining the end of a measurement by a limiter (or a game).

FIG. 18 is a diagram describing an example of a method of interrupting a game.

FIG. 19 is a diagram describing a living body input device and living body control device using an optical instrumentation method for the living body.

### DETAILED DESCRIPTION OF THE PREFERRED EMBODIMENTS

According to this invention, the activity of a localized brain function is measured using light, and the measured signal is used as, for example, an input signal to a computation unit of a computer. Specifically, at least one light illumination device and at least one light detector are brought in contact with the skin of one or more subjects, and the intensity variation of the light transmitted through the living body is measured. This measurement result is reflected in the concentration variation of a metabolite (oxidized hemoglobin or reduced hemoglobin, etc.) in the living body accompanying the brain activity. The measurement result is used as an input signal to the computation unit. A display unit connected to the computation unit comprises a screen for displaying (presenting) information to the subject, there being at least one object on the screen. The form (position, color, size) of the one or more objects varies according to a concentration or concentration variation of the metabolite in the living body, i.e, the variation of an input signal intensity (or intensity variation of light transmitted through the living body) which reflects this concentration or concentration variation.

Specifically, this permits construction of an amusement system which measures a brain function using light, wherein the o0 state of an object on the screen connected to the computation part of the computer varies according to the activation state of the brain function.

Hereafter, specific examples of this invention will be described referring to the drawings.

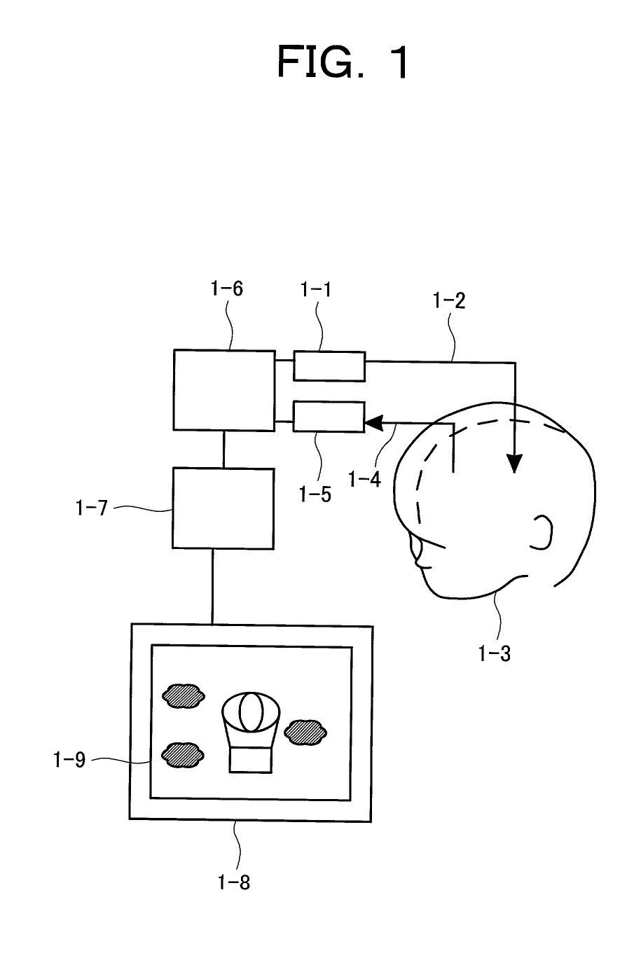

FIG. 1 shows an example of an amusement system wherein a concentration or concentration variation of a metabolite (e.g., oxidized hemoglobin, reduced hemoglobin or chitochromes in blood) in a living body is measured using at least one light illumination means and at least one optical detection means on one player, and brain activity depending on the light intensity propagated in the living tissue is reflected in an object displayed on the screen. 1-1 is a light source such as a semiconductor laser, photodiode or lamp, and its end is connected with a light waveguide (e.g., optical fiber) 1-2.

The other end of the optical fiber comes in contact with the skin (e.g., scalp) of the subject (1-3). When it contacts the scalp, it is preferable to push the hair on the scalp away so that the end of the light waveguide comes in direct contact with the scalp. This is because, if the light used for measurement is absorbed by the hair, light illumination efficiency falls.

1-4 is an optical fiber used for detecting light irradiated by the light waveguide (1-2) which is propagated through a subject (103). One end of this optical fiber (1-4) comes in contact with the skin (e.g., scalp) of the subject (1-3). For the same reason, regarding this optical fiber also, it is preferable to push the hair on the scalp away so that the end of the light waveguide comes in direct contact with the scalp. The other end is connected to a light detector (1-5) such as an avalanche photodiode or photomultiplier. This light source (1-1) and light detector (1-5) are electrically connected to a controller (1-6). This controller (1-6) is connected to an electronic calculator (1-8) such as a computer or the like via an analog/digital converter (1-7).

Information can be transmitted in both directions between this controller (1-6) and the electronic calculator (1-8). The controller (1-6) permits adjustment of the light amount from the light source (e.g., switching the light source ON or OFF, generating a pulse-like light source, or adjusting the emission intensity of the light source with an angular frequency).

The electronic calculator (1-8) comprises a storage device (e.g., hard disk or memory) for temporarily or permanently storing information concerning the intensity variation (time dependency) of the detected light. The analog light intensity reaching the light detector (1-5) is converted to a digital light intensity via the analog/digital converter (1-7), and the result is transmitted to the electronic calculator (1-8).

A signal which commands the light amount adjustment of the light source is transmitted from the electronic calculator (1-8) to the controller (1-6). According to this embodiment, the controller (1-6), analog/digital converter (1-7) and electronic calculator (1-8) are implemented as separate devices, but there is no problem if they are integrated in one device.

The electronic calculator (1-8) comprises a display unit provided with a screen (1-9) to implement an amusement system which reflects brain activity depending on the light intensity transmitted through the living body in an object displayed on the screen. The construction of this screen (1-9) will be described using another embodiment. This display unit may be formed in one piece together with the computation unit, or formed as a separate unit.

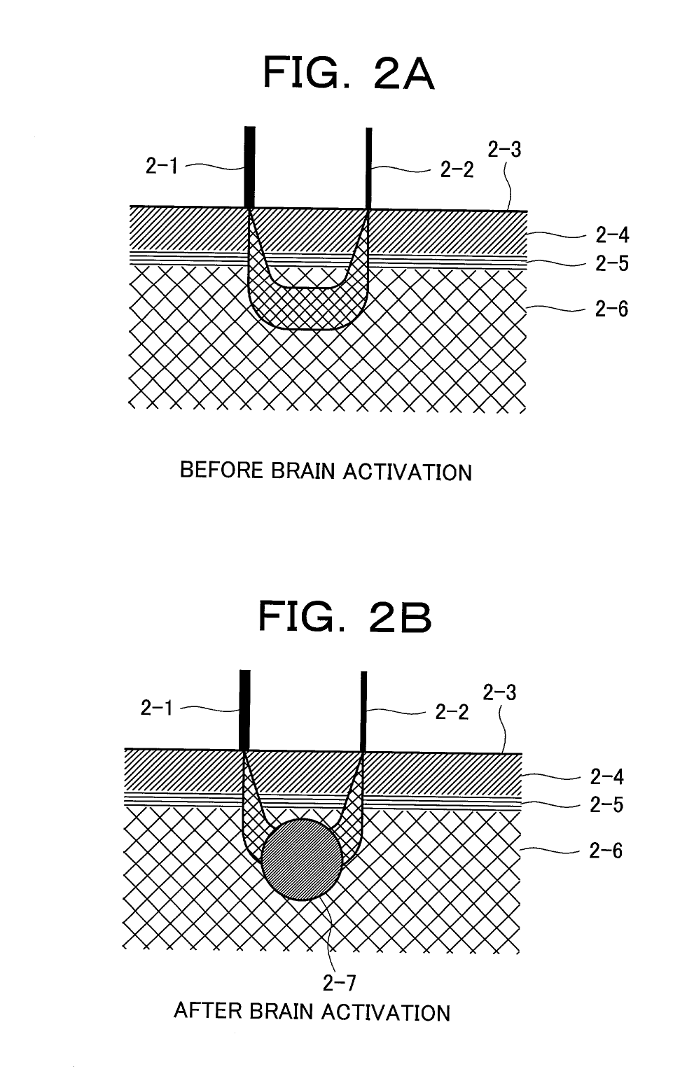

Next, a method for measuring the variation of metabolite concentration in the living body using the instrumentation method shown in FIGS. 2A and 2B, will be described.

First, an light illumination waveguide (2-1) and optical detection waveguide (2-2) are brought in contact with the scalp of a subject (2-3). In the human brain, the skull (2-4), cerebrospinal fluid layer (2-5) and the cerebral cortex (2-6) exist in layers under the scalp. Here, the light illumination waveguide (2-1) and optical detection waveguide (2-2) are brought in contact with the scalp lightly so that the subject (2-3) does not feel pain.

The living tissue strongly scatters the light. As a result, part of the scattered light passes through the cerebral cortex (2-6) inside the skull (2-4) in which individual high level brain functions are concentrated, and reaches the contact point between the light waveguide (2-2) for detection and the scalp of the subject (2-3), as shown in FIG. 2A. In the case of an adult, this final point is generally approximately 30 mm from the light illumination position (contact point on the scalp of the light waveguide (2-1) for illumination).

Here, when the brain is active, the blood volume (oxidized hemoglobin, reduced hemoglobin concentration) in the cerebral cortex varies to supply oxygen and glucose to active sites in the brain nerve cells (2-7), as shown in FIG. 2B. For this measurement, it is preferable to use near infrared light (wavelength: approximately 800 nm) which has a high transmittance in living tissue (difficult to measure for water or protein in the living body), and which is absorbed by hemoglobin in the blood (oxidized hemoglobin, reduced hemoglobin). The invention is of course not limited to light of this wavelength. Here, when the blood volume increases (decreases) in the cerebral cortex due to brain activity, the detected light intensity decreases (increases).

Next, an example of an amusement system using the measurement system shown in FIG. 1 which reflects brain activity (nervous activity) dependent on the light intensity propagated in living tissue relative to an object displayed on the screen, will be described.

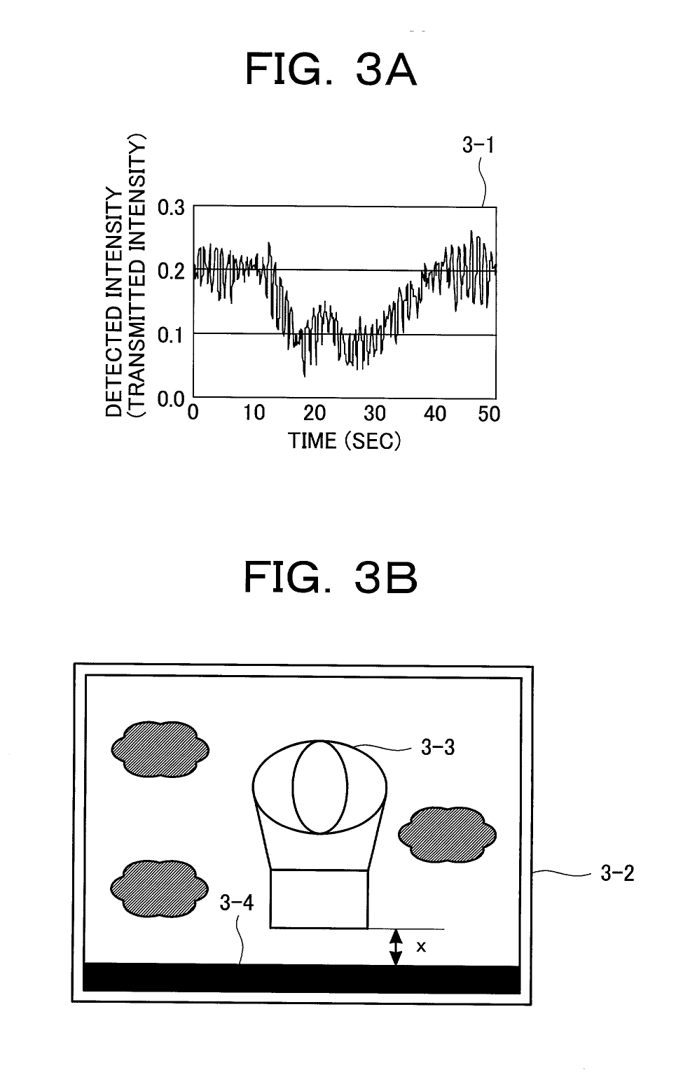

First, 3-1 in FIG. 3A shows an example of a relation between the intensity of the light transmitted through living body tissue and measurement time. In this measurement, the optical fiber for illumination and the optical fiber for detection shown in FIG. 1 were disposed at an interval of 30 mm on the “forehead” 1 cm above the left eyebrow of the subject. During the measurement interval (50 seconds) in the figure, the subject repeatedly opened and shut his right hand from 10 seconds to 30 seconds (20 second interval) with a frequency of 1 Hz. However, during these intervals, the subject remained quiet. From the measurement result, it is seen that the detected light intensity several seconds after starting the task decreases, and the detected light intensity increases when the task is finished. This corresponds to the fact that metabolite concentration (hemoglobin concentration) in the living body increases due to brain activity.

The example shown in 3-2 of FIG. 3B was therefore proposed. In this example, there is a balloon (3-3) on the screen. This balloon is at a height x above the ground (3-4).

This height x may be determined according to example (3-1) from the relation between the intensity of the light transmitted through the living tissue and measurement time. The detected light intensity at the time t=0 in equation (1) is a certain reference intensity, which maybe the intensity at any time during the measurement interval (during operation of the amusement system), the average value during this interval or a reference intensity determined at any other time. The determination method is of course not limited to the method using equation (1).

x=ln {(detected light intensity at t=0)/(detected light intensity at time t)} (1)

According to this equation (1), x increases as the transmitted light intensity decreases, and x decreases as the transmitted light intensity increases. Therefore, the height of the balloon increases when there is brain activity. By using the above method, firstly, when the brain becomes active when the subject thinks of something, concentrates on something or attempts to move the hand or foot, an invisible human brain function is measured, secondly, the result is directly input to an electronic calculator, and thirdly, this human brain function is visualized. In other words, an amusement system can be provided wherein the object displayed on the screen changes according to brain activity.

A modification of the above example is described below.

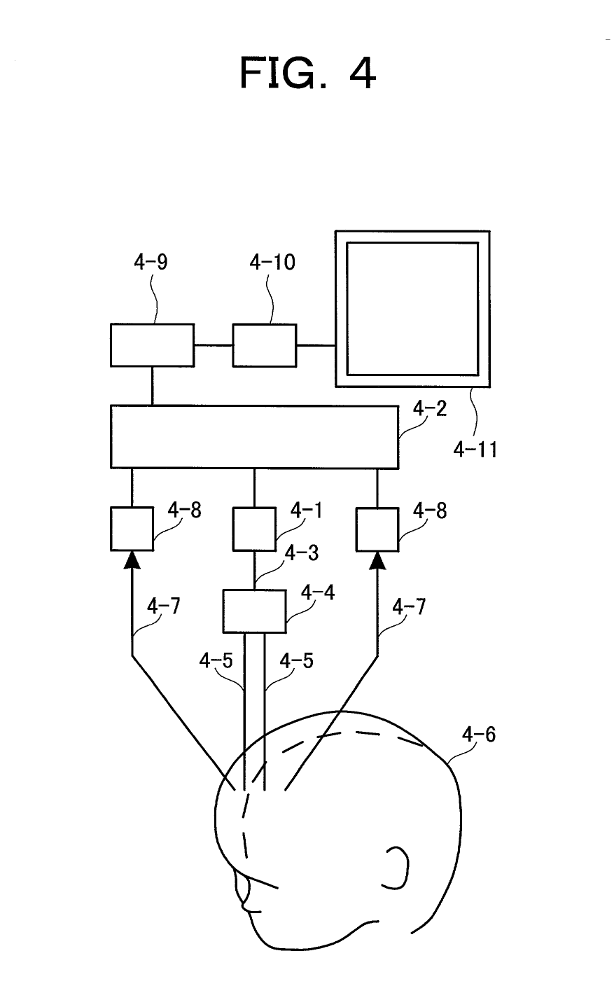

In FIG. 4, plural measurement points are set on the scalp of the subject, and the concentration variation of the metabolite at these plural measurement points is measured. In this example, an amusement system is realized which reflects brain activity dependent on the light intensity propagated in living tissue relative to the object displayed on the screen based on the measurement result of this concentration variation. 4-1 is a light source such as a semiconductor laser, photodiode or lamp. This emitted light intensity is controlled by a controller (4-2) 4-1 and 4-2 being electrically connected.

The other end of the light source (4-1), i.e., the semiconductor laser, photodiode or lamp, is connected to a light waveguide (4-3) such as an optical fiber, and this optical fiber is further connected to a light waveguide (4-5) for illumination such as plural optical fibers by an optical coupler (4-4). The ends of these plural optical fibers are connected at plural points on the scalp of a subject (4-6). For example, they maybe disposed on the left and right temporal lobes. The invention is of course not limited to this arrangement, and the fibers may be disposed at any desired points on the scalp of the subject. Light waveguides (4-7) such as optical fibers for detection are disposed at points several cm from the ends of the light waveguides (4-5) for illumination which may also be optical fibers. For example, if the measurement target is the concentration variation (blood volume variation) of a metabolite in the living body due to human brain activity, it is desirable to place the fibers at about 3 cm away, but the invention is of course not limited to this value.

The other ends of the light waveguides (4-7) which may be optical fibers used for detection, are connected to light detectors (4-8) such as photomultipliers. Due to these light detectors (4-8), the light intensity propagated in the living body is converted to an electrical signal intensity. This is input to the controller (4-2) which is electrically connected. The signal intensity input to the controller (4-2) is input to an analog/digital converter (4-9), and this digital signal is input to a computation unit (display controller) (4-10). According to this embodiment, this display controller (4-10) comprises a display screen (4-11). The content displayed on the screen will be described later. Using the above measurement method, it is possible to produce an amusement system which reflects brain activity dependent on light intensity propagated in the living body relative to an object displayed on the screen, described hereafter.

The light waveguides (4-5) for illumination such as the optical fibers shown in FIG. 4 are disposed at plural points on the scalp. Human brain function is localized in the cerebral cortex for each function. For example, if one of these light waveguides is positioned on the left temporal lobe and the other is positioned on the right temporal lobe, the brain activity of the right finger movement field and left finger movement field can be respectively measured. In other words, the left temporal lobe can be activated by moving the fingers of the right hand, and as a result, the activation of the brain in the left temporal lobe can be measured using the light waveguide (4-5) for illumination and light waveguide (4-7) such as an optical fiber for detection positioned on the scalp of the subject (4-6). Likewise, the right temporal lobe can be activated by moving the fingers of the left hand, and as a result, the activation of the brain in the right temporal lobe can be measured using the light waveguide (4-5) for illumination and light waveguide (4-7) such as an optical fiber for detection positioned on the scalp of the subject (4-6). Hence, various types of brain activity can be measured by installing plural measurement points.

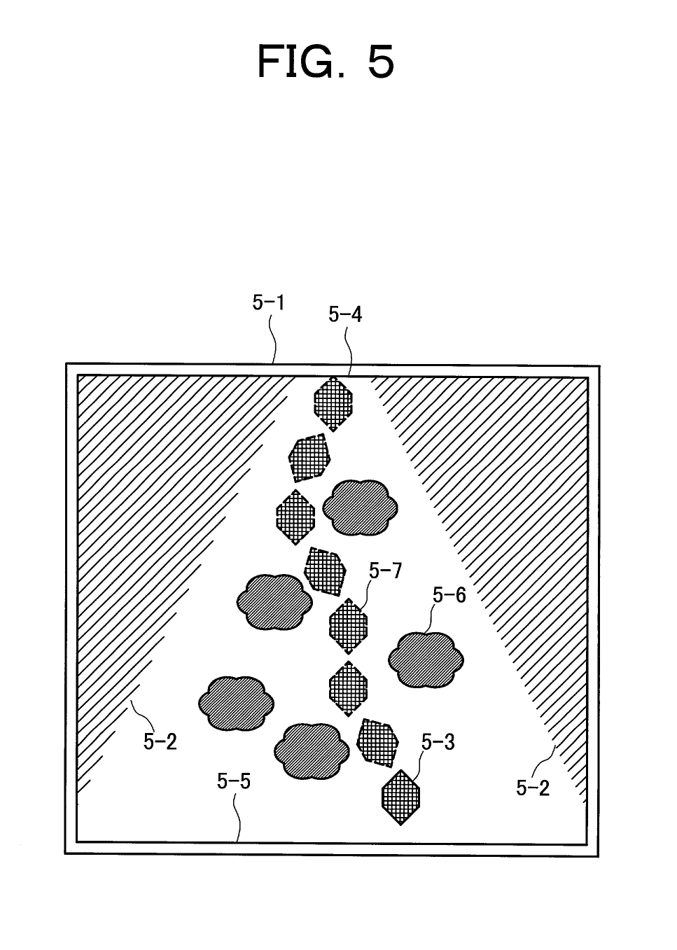

In this connection, a method of constructing an amusement system which reflects brain activity dependent on the light intensity propagated in living tissue relative to an object displayed on the screen (4-11), will be described referring to FIG. 5. 5-1 is a display screen, and on this display screen, a display symbolizing a canoe sailing down a river is shown. 5-2 are the riverbanks, and the canoe (5-3) navigates the middle of the river between the riverbanks. This canoe navigates from the upper reaches (5-4) to the lower reaches (5-5) of the river according to the flow velocity at each point prestored in the electronic calculator.

5-6 in FIG. 5 are obstacles, and when these obstacles touch the canoe (5-4), the canoe no longer moves. The obstacles denoted by 5-6 for example suggests rocks or flotsam in the river and are not limited to the shape of the obstacles shown in FIG. 5. The canoe (5-7) with the dotted line shape shown in FIG. 5 is the position of the canoe in the river within the time sequence, and it moves down the river without touching the obstacles (5-6) In order to proceed down the river in this way, it is convenient to independently activate the finger movement fields in the left and right temporal lobes by moving the left and right fingers.

As a result, an amusement system which reflects brain a activity dependent on light intensity propagated in living tissue relative to an object displayed on the screen (in the case of FIG. 5, the canoe (5-3)), can be constructed.

The brain function measurement method using light described above has a superior feature not found in brain function measurement methods using magnetism (e.g., a functional magnetic drawing device) or brain function measurement methods using radiation (e.g., a positive electrode illumination tomography apparatus). This is that although the device is compact, localized human brain functions can be measured safely. Consequently, the human brain functions of plural subjects can also be simultaneously measured. In this regard, an example of an amusement system which simultaneously measures the human brain functions of plural subjects, and reflects brain activity dependent on light intensity propagated in living tissue relative to an object displayed on the screen, will now be described.

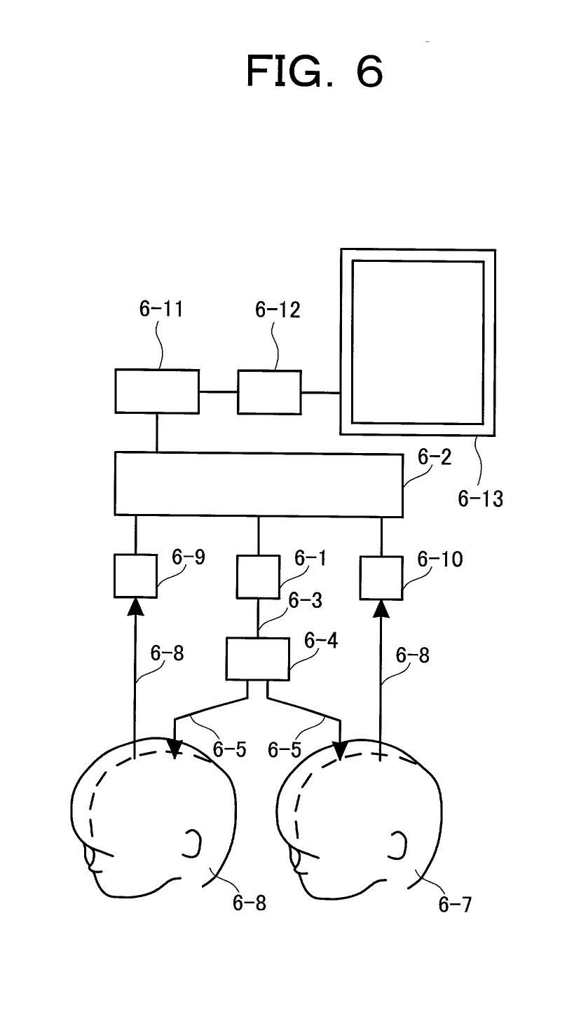

FIG. 6 is one form of an embodiment where plural subjects are simultaneously measured. 6-1 is a light source such as a semiconductor laser, photodiode or lamp. The emitted light intensity is controlled by a controller (6-2), 6-1 and 6-2 being electrically connected. The other end of the light source (6-1) i.e., the semiconductor laser, photodiode or lamp, is connected to a light waveguide (6-3) which may be an optical fiber, and this optical fiber is connected to light waveguides (6-5), which may be plural optical fibers, by an optical coupler (6-4). The ends of these plural optical fibers are respectively brought in contact with the scalps of a subject 1 (6-6) and a subject 2 (6-7).

Light waveguides (6-8) such as optical fibers used for detection are disposed at points several cm away from the ends of the light waveguides (6-5) used for illumination, which may be optical fibers. For example, if the measurement target is the concentration variation (blood volume variation) of a metabolite in the living body due to the activity of a human brain function, it is preferable to position them at about 3 cm away, but the invention is of course not limited to this value.

The other ends of the light waveguides (6-8), which may be optical fibers used for detection, are connected to optical detectors (6-9 and 6-10) such as avalanche photodiodes or photomultipliers. Due to this light detector, the intensity of light propagated in the living body is converted to an electrical signal intensity. This is input to the controller (6-2) which is electrically connected. The signal intensity input to the controller is input to an analog/digital converter (6-11), and this digital signal is then input to a display controller (6-12). This display controller comprises a display screen (6-13). The contents displayed on the screen will be described later.

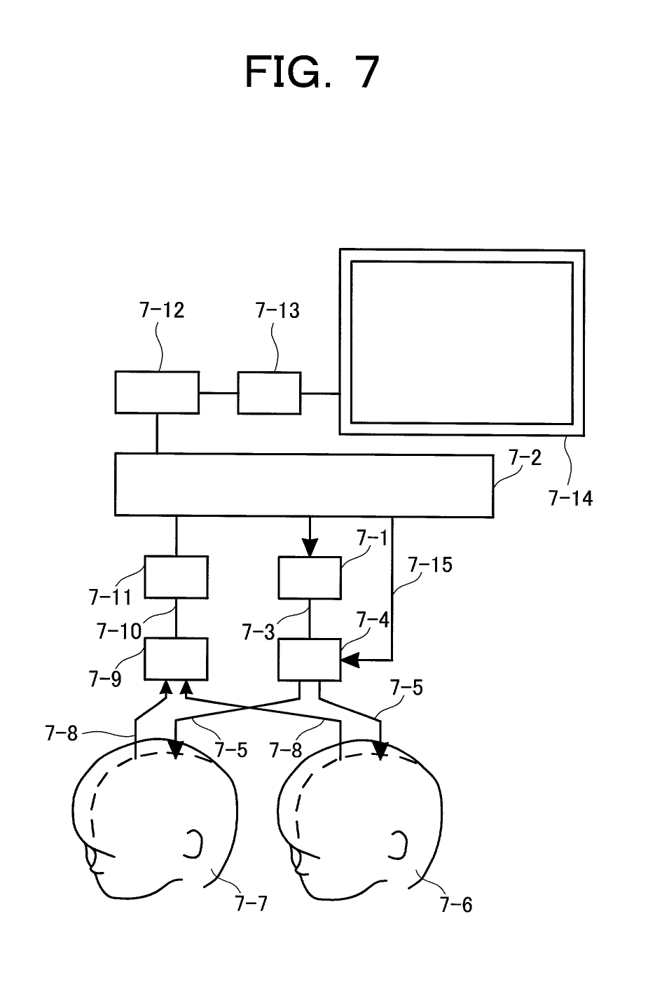

In the above measurement method, the light detectors (6-9 and 6-10) such as avalanche photodiodes or photomultipliers had to be installed at plural points. In this connection, the following example of a measurement method wherein plural subjects can be simultaneously measured by a smaller number of detectors will now be described referring to FIG. 7. 7-1 is a light source such as a semiconductor laser, photodiode or lamp. The emitted light intensity is controlled by a controller (7-2), 7-1 and 7-2 being electrically connected.

The other end of the light source (7-1), i.e., the semiconductor laser, photodiode or lamp, is connected to a light waveguide (7-3) such as an optical fiber, and this optical fiber is further connected to light waveguides (7-5) such as plural optical fibers by an optical coupler (7-4). The ends of these plural optical fibers are respectively brought into contact with the scalps of a subject 1 (7-6) and a subject 2 (7-7).

Light waveguides (7-8) such as optical fibers for detection are disposed at points several cm away from the ends of the light waveguides (7-5) used for illumination which may also be optical fibers. For example, if the measurement target is the concentration variation (blood volume variation) of a metabolite in the living body due to the activity of a human brain function, it is preferable to position them about 3 cm away, but the invention is of course not limited to this value. The other ends of the light waveguides (7-8) which may be optical fibers used for detection are connected to an optical coupler (7-9).

The output of this optical coupler is connected to a light detector (7-11) such as an avalanche photodiode or a photomultiplier via a light waveguide (7-10) which may be an optical fiber. Due to this light detector, the intensity of the light propagated in the living body is converted to an electrical signal intensity. This is input to the controller (7-2) which is electrically connected. The signal intensity input to the controller is input to an analog/digital converter (7-12), and this digital signal is input to a display controller (7-13). This display controller comprises a display screen (7-14). The optical coupler (7-4) is connected to the controller (7-2) via a control command transmission circuit (7-15).

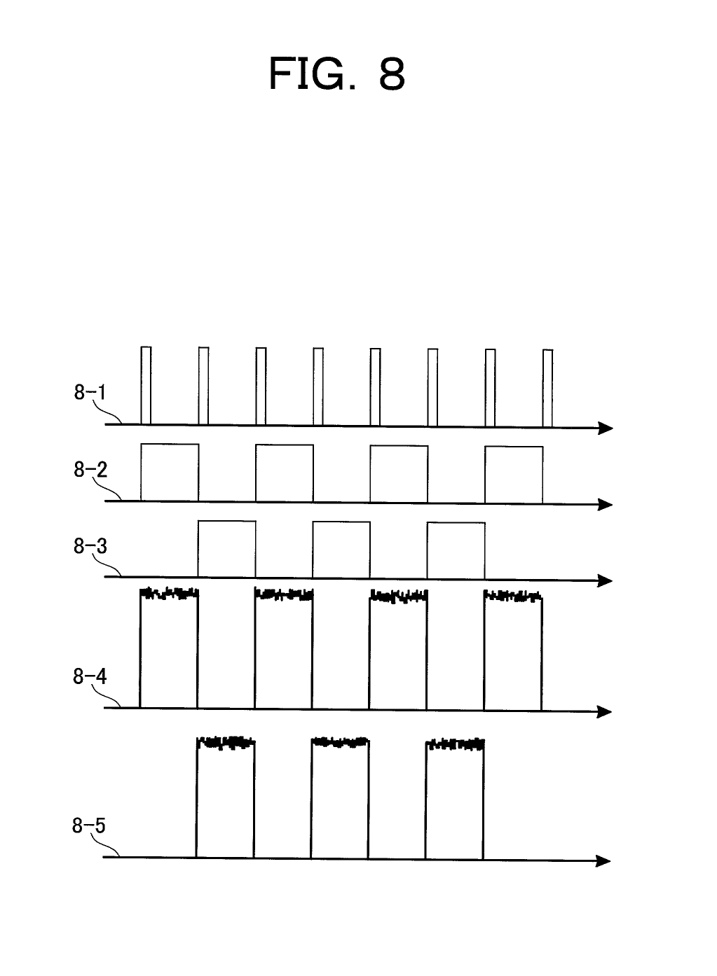

In the measurement method shown in FIG. 7, light from one light source (7-1) irradiates plural subjects (7-6, 7-7), and the light intensity propagated through each subject is measured using one light detector (7-11). The control sequence shown in FIG. 8 below is provided to clarify the question of through which subject the detected light has propagated. 8-1 is a control pulse emitted from the controller (7-2) to the optical coupler (7-4). The illumination interval of this pulse may for example be 100 msec, but the invention is of course not limited to this value.

When this control pulse is received by the optical coupler (7-4), the light intensities irradiating the subject 1 (7-6) and subject 2 (7-7) are interchanged via the optical fibers (7-5) for illumination, as shown in 8-2 and 8-3. 8-4 and 8-5 are the light intensities propagated in the subject 1 (7-6) and subject 2 (7-7) detected by the light waveguides (7-8) which may be optical fibers for detection, and can be detected in synchronism with the time dependency of the light intensity shown by 8-2 and 8-3. These light intensities are converted to electrical signals by the light detector (7-11) via the optical coupler (7-9). In the controller, the conversion results may be distinguished for each light intensity transmitted through the subjects (7-6, 7-7) in synchronism with the control pulse (8-1) emitted from the controller (7-2) to the optical coupler (7-4).

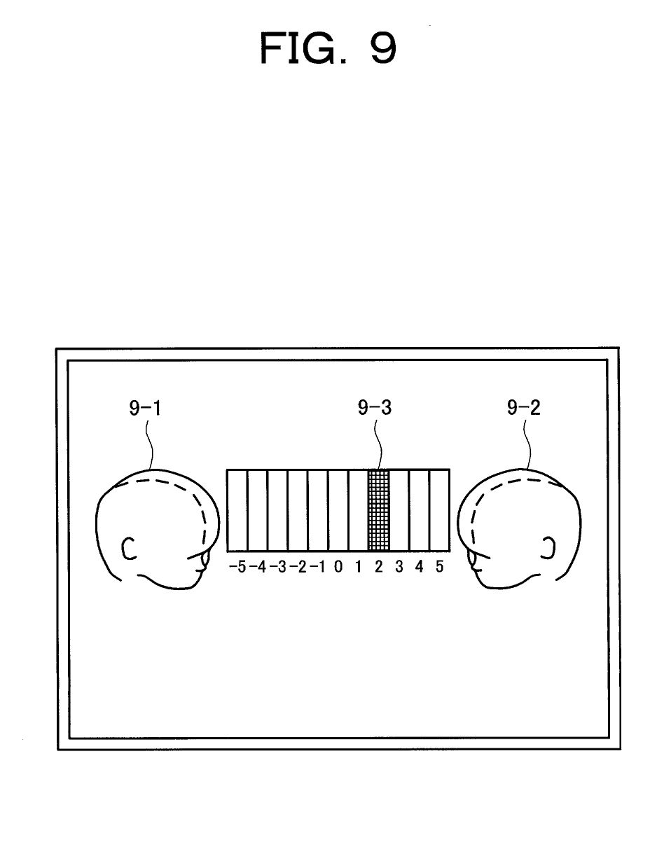

Using the measurement method shown in FIG. 6 or FIG. 7, a method of realizing an amusement system which reflects a brain activity depending on the light intensity propagated in living tissue relative to an object displayed on the display controller (6-13 in FIG. 6 or 7-14 in FIG. 7) will now be described referring to FIG. 9.

FIG. 9 shows a tug-of-war contest between a subject 1 (9-1) and a subject 2 (9-2). The blood volume change due to brain activity of the subjects (subject 1 and subject 2) may for example be calculated by the computation method shown in equation 1. For example, in the case of a blood volume change (B1) of subject 1 (9-1) and a blood volume change (B2) of subject 2 (9-2), the difference between the blood volume changes is given by (B1-B2). 9-3 in FIG. 9 is an indicator showing this difference, this figure showing the case where B1-B2=2.

In this figure, the variation range of B1-B2 is from −5 to +5, but the invention is of course not limited to this range. The brain function is different for each subject and each measurement site (each point where the illumination optical fiber and detection optical fiber are installed). As a result, an amusement system can be realized which reflects brain activity dependent on the light intensity propagated in living tissue relative to an object displayed on the display controller (6-13 in FIG. 6 or 7-14 in FIG. 7).

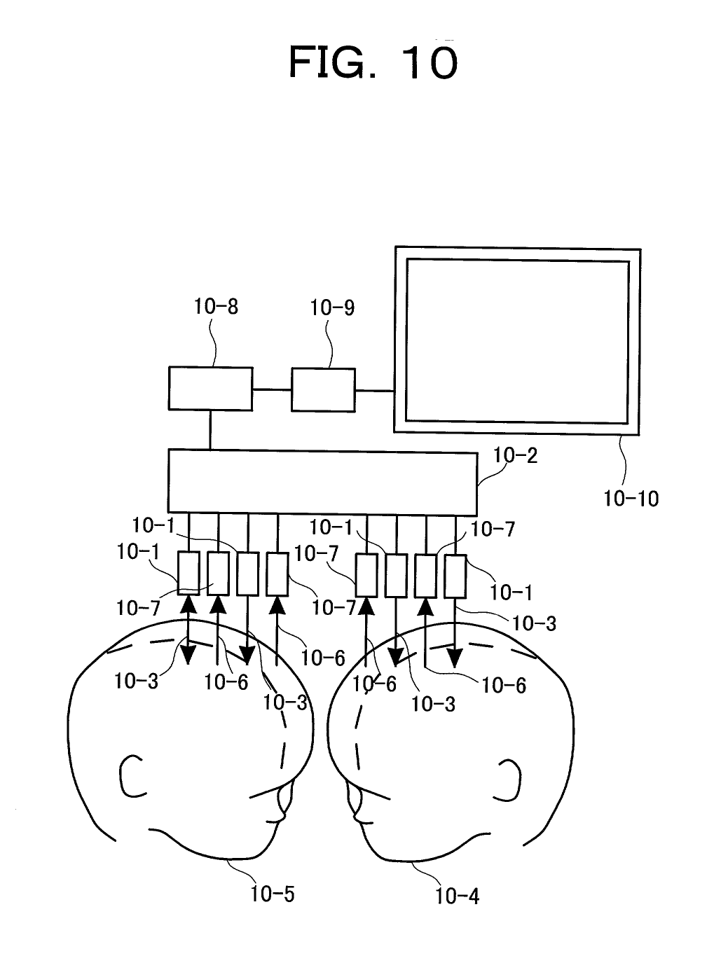

FIG. 10 is a modification of the measurement method shown in FIG. 4 and FIG. 6. 10-1 are light sources such as semiconductor lasers, photodiodes or lamps. The emitted light intensity is controlled by a controller (10-2), 10-1 and 10-2 being electrically connected. The other ends of the light sources (10-1), i.e., the semiconductor lasers, photodiodes or lamps, are connected to light waveguides (10-3) which may be optical fibers. The ends of the plural optical fibers are connected to the scalps of the subject 1 (10-4) and subject 2 (10-5), two at a time.

Light waveguides (10-6) such as optical fibers for detection are disposed at positions several cm from the ends of the light waveguides (10-3) used for illumination which may also be optical fibers. For example, if the measurement target is a concentration variation (blood volume variation) of a metabolite in the living body due to the activity of a human brain function, it is preferable that they are positioned about 3 cm away, but the invention is of course not limited to this value.

The other ends of these light waveguides which may be optical fibers for detection are connected to light waveguides (10-7) such as avalanche photodiodes or photomultipliers. Due to these light waveguides (10-7), the intensity of the light propagated in the living body is converted to an electrical signal intensity. This is input to the controller (10-2). The signal intensity input to the controller is input to an analog/digital converter (10-8), and this digital signal is input to a display controller (10-9). This display controller comprises a display screen (10-10).

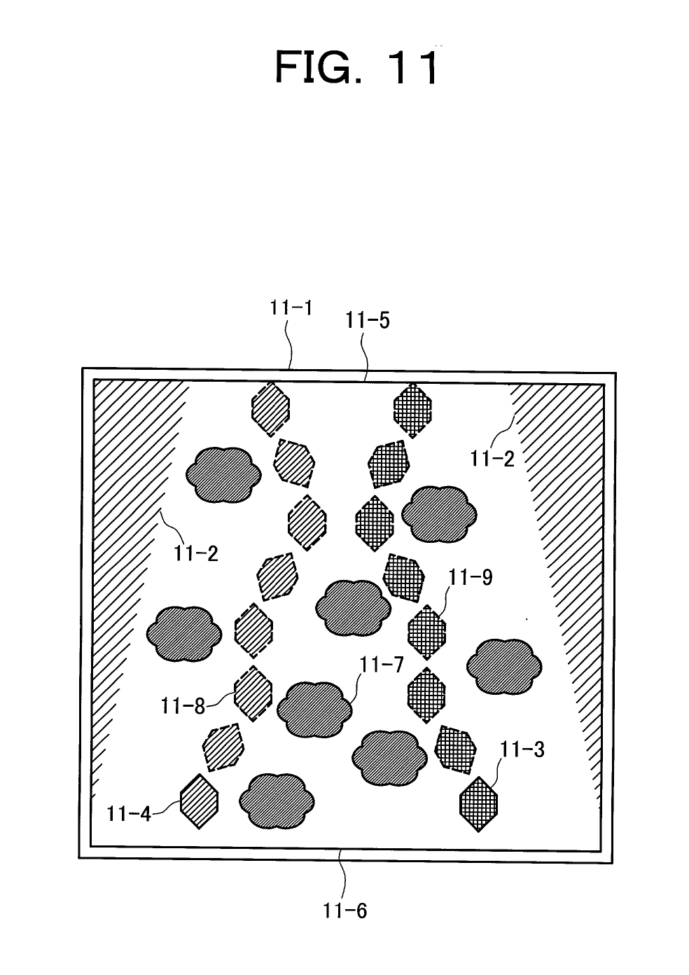

Next, an example of the contents displayed on the display screen (10-10) of FIG. 10 will be described. The fiber used in the example of FIG. 10 may for example be disposed on the left and right temporal lobes (on the movement field). As described above, when the fingers of the left and right hands are moved, for example, the left and right movement fields can be activated independently. Hence, the intention of the subject can be reflected in an object displayed on the screen by activating the left and right movement fields. As plural subjects are being measured, the subjects can engage in a competition. An example where this competition amusement system is implemented will be described referring to FIG. 11.

11-1 is a display screen, and on this display screen, a journey down river is symbolized by a canoe. 11-2 are riverbanks, and a canoe 1 (11-3) and canoe 2 (11-4) navigate the river enclosed between the riverbanks. These canoes sail from the upper reaches (11-5) to the lower reaches (11-6) of the river according to the flowrate of the river at each point prestored in an electronic calculator. 11-7 are obstacles, and when the canoes (11-3, and 11-4) come in contact with these obstacles, the canoes can no longer move (the flowrate in the flow direction at this point is zero).

The obstacles shown by 11-7 may for example be rocks or flotsam in the river, and are not limited to the shape of the obstacles shown in FIG. 11. The canoe 1 (11-8) and canoe 2 (11-9) with the dotted line shape shown in FIG. 5 are the positions of the canoes in the river within the time sequence, and they move down the river without coming in contact with the obstacle (11-7). The finger movement areas on the left and right temporal lobes may be independently activated by moving the left and right fingers in order to make the canoes move down the river in this way.

As a result, an amusement system which reflects brain activity dependent on the light intensity propagated in living tissue, in the object (in the case of FIG. 11, the canoe (11-3 and 11-4)) displayed on the screen, can be realized.

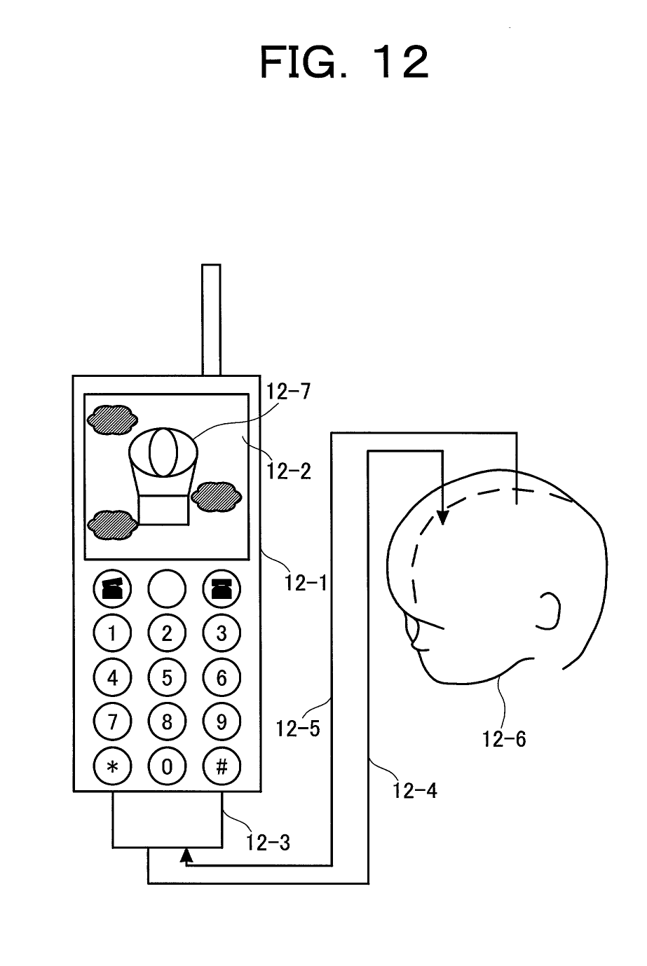

In the living body instrumentation method using light, a semiconductor light source such as a semiconductor laser or photodiode and a semiconductor detector such as a photodiode can be used, so the device can be made compact. This compact instrumentation device and an example of an amusement system which reflects brain activity dependent on light intensity propagated in living tissue in an object displayed on a screen, are shown in FIG. 12.

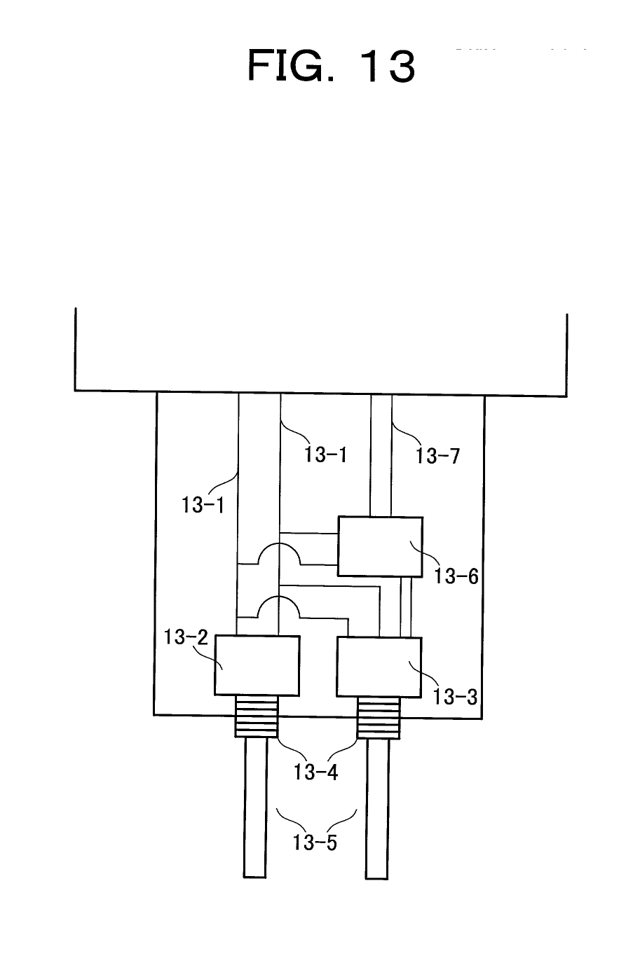

In FIG. 12, 12-1 is an information terminal comprising a display screen 12-2. A controller (12-3) comprising a light source and detector is connected below this information terminal. An example of the internal structure of this controller will be described using FIG. 13. An optical fiber (12-4) for illumination and optical fiber (12-5) for detection are connected to one end of this controller. The ends of these optical fibers lightly touch the scalp of a subject (12-6). An object (12-7) reflecting brain activity is displayed on the display screen. According to this embodiment, a balloon is displayed whose height varies according to the amount of blood volume change due to brain activity, but the invention is of course not limited to a balloon. This object may be displayed using the example described in FIG. 3. The invention is of course not limited to this display method.

Next, the internal construction of the controller shown by 12-3 in FIG. 12 will be described using FIG. 13. 13-1 is a power cable, and this runs from the information terminal (12-1) shown in FIG. 12. This power cable is used to control a light source (13-2) such as a semiconductor laser or photodiode, a detector (13-3) such as an avalanche photodiode or photomultiplier, and an analog/digital converter (13-6). The light source and light detector are connected to light waveguides (13-5) such as optical fibers via optical fiber connectors (13-4) The detector (13-3), i.e., avalanche photodiode or photomultiplier, converts the light intensity transmitted in the living body tissue which has propagated through the light waveguide (13-5), which may be an optical fiber, into an electrical signal, and this is digitized by the analog/digital converter (13-6). The result is transmitted to the information terminal (12-1) by a signal transmission cable (13-7).

In the example described above, a metabolite concentration or its concentration variation in living tissue reflecting a mental state was measured by light, and the measurement result was reflected in positional information relating to an object displayed on a screen. In addition to this reflection method, other reflection methods shown below are also possible.

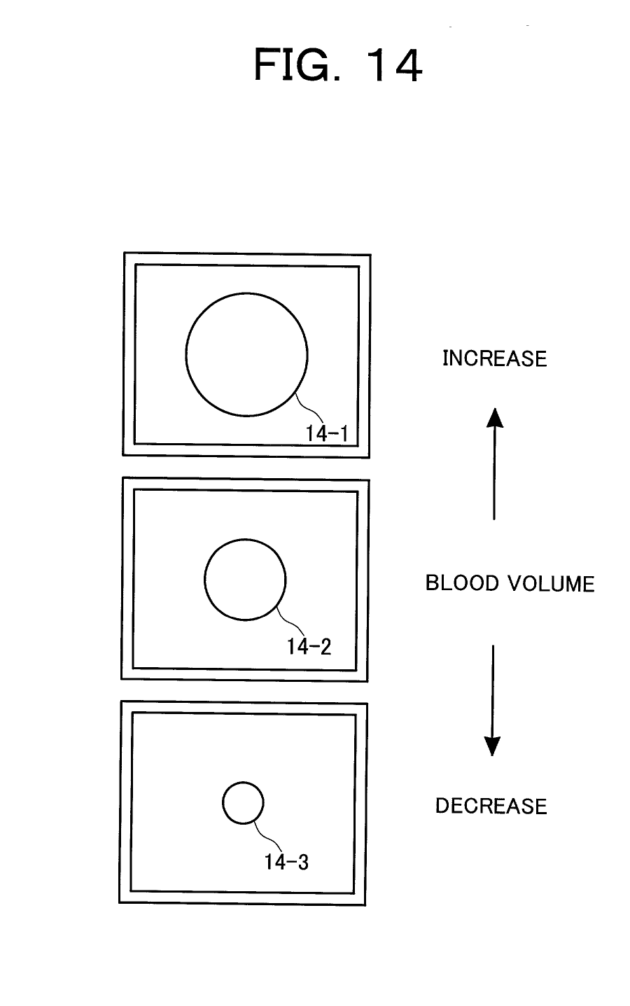

FIG. 14 is an example of a method wherein the variation (increase or decrease) of blood volume due to brain activity is displayed by varying the size of an object on the screen.

In FIG. 14, 14-2 is the size of the object at a reference blood volume.

14-1, 14-3 shows the size of the object when blood volume is increased or decreased relative to the reference blood volume. As a specific example of the content displayed, if blood volume in the brain can be increased by concentrating on the idea “grow” applied to the object displayed on the screen (e.g., the human body or a frog's stomach), the size of the object will increase. If the measurement position is offset, however, blood volume may actually decrease, and in this case, the size of the object will decrease.

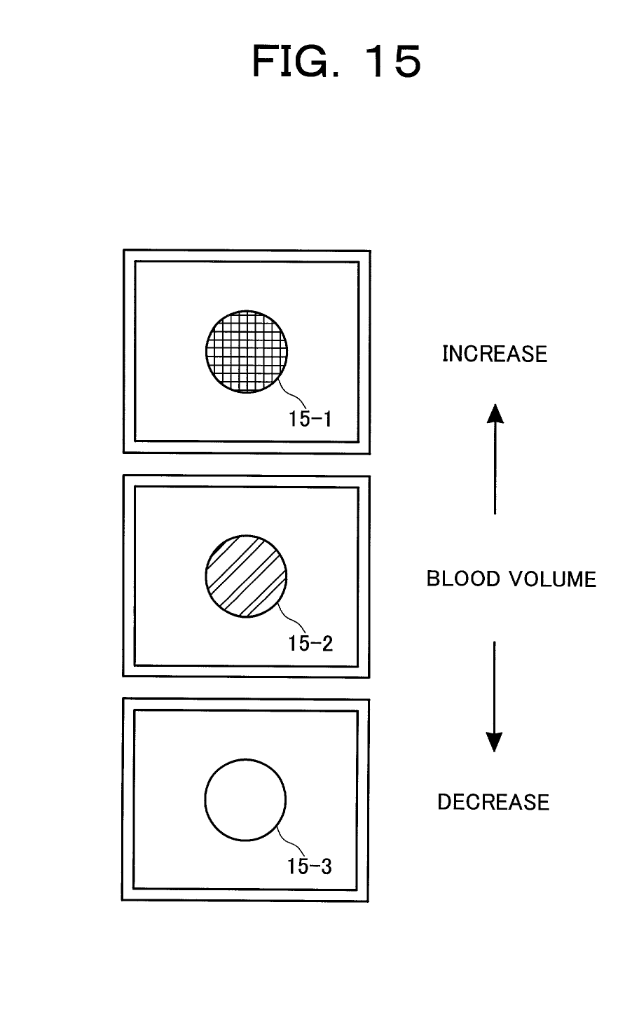

FIG. 15 shows an example where blood volume variation (increase or decrease) due to brain activity is displayed by varying the color (light and shade, or type) of the object on the screen. 15-2 is the color (e.g., red) of the object at the reference blood volume. On the other hand, 15-1, 15-3 respectively show the color of the object when blood volume increases and decreases relative to the reference blood volume. 15-1 shows the case where the basic redness changes to crimson due to an increase of blood volume, and 15-2 shows the case where it changes to pink due to a decrease of blood volume.

Further, the basic red color may also for example be changed to blue when blood volume increases, and changed to yellow when it decreases. As a specific example of this content, if blood volume in the brain can be increased by concentrating on the idea “turn blue ” regarding the object (a signal which is red) displayed on the screen, the color of the object will turn blue. When the blood volume decreases, the color of the object will turn another color (e.g., yellow).

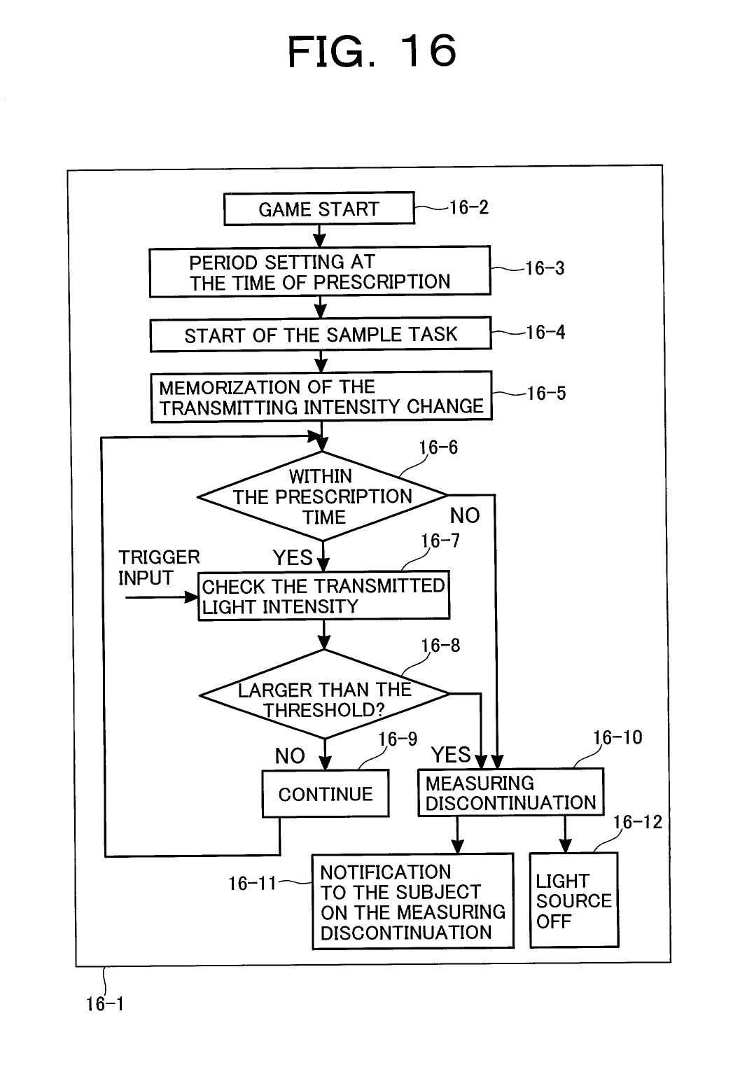

Next, an example of an amusement system (limiter) which does not easily cause fatigue in a subject will be described. 16-1 in FIG. 16 shows this example as a flowchart. The algorithm based on this flowchart may for example be stored in a storage device in the electronic calculator shown by 1-8. An outline of the flowchart shown in 16-1 is described below.

First, a game is started (16-2). A specific time is then set (16-3), and stored in the storage device. This specific time is set to, for example, 30 minutes. This is because a player generally becomes very involved in a game and tends to forget the passage of time. As a result, he plays the game for endless hours and becomes fatigued without noticing it. This specified measurement time may be set arbitrarily according to the type of game and individual endurance.

Next, a sample task is performed (16-4). This is done by giving the subject a message such as “move your hand” or “think of something you enjoyed”, and measuring the variation of light intensity transmitted in the body of the subject using the light illumination device and light detector positioned on the subject. The variation of detected light intensity when the subject is resting is taken as x, and this is also stored in the storage device (16-5). Further, a threshold parameter k for the variation of transmitted light intensity is set in this storage device. This threshold value can also be arbitrarily set according to the type of game and individual endurance.

The main game is then undertaken. First, the cumulative measurement time from when the game was started is calculated. If the cumulative measurement time is within the specified measurement time (reference illumination interval), measurement is continued, otherwise measurement (the game) is stopped (16-6). Next, the variation of transmitted light intensity is checked (16-7), and it is determined whether the intensity is larger or smaller than the set threshold value (x×k) (16-8). When the variation of transmitted light intensity is less than the set threshold value, the game is continued (16-9). On the other hand, when it is greater than the threshold value, the game is stopped (16-10). An instruction to stop the game is then given to the subject (16-11), and the power is switched OFF (16-12).

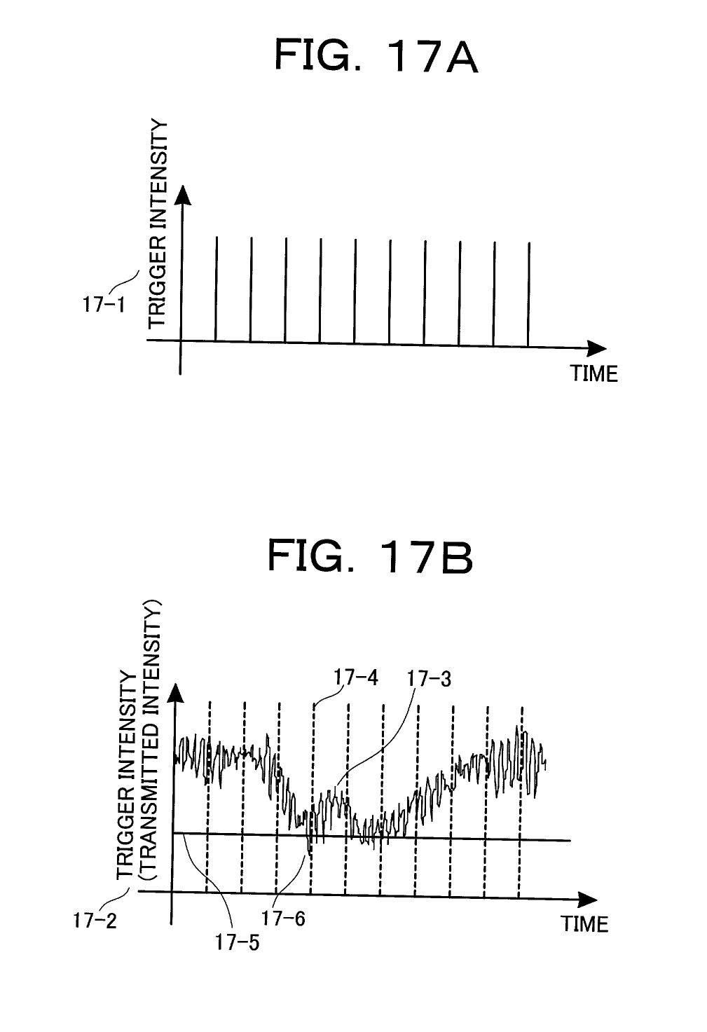

Next, a method of checking the variation of transmitted light intensity will be described below referring to FIGS. 17A and 17B. First, a trigger is emitted to examine the detected light intensity (transmitted light intensity) according to a certain time interval, as shown by 17-1 in FIG. 17A. This trigger generation interval is not limited to the equal intervals shown in the figure, and may be set to arbitrary intervals. The light intensity transmitted in living tissue (detected light intensity) input to the electronic calculator is then checked corresponding to this trigger.

17-2 in FIG. 17B shows an example of this checking method. 17-3 shows the time dependency of the light intensity transmitted in living tissue. 17-4 shows the timing of the trigger shown by 17-1 superimposed on 17-2. Further, 17-5 shows a certain predetermined threshold value intensity. This threshold value intensity is set to an arbitrary value for each subject or each amusement system. In the case of 17-2, in an interval 17-6, the transmitted light intensity falls below the threshold value intensity.

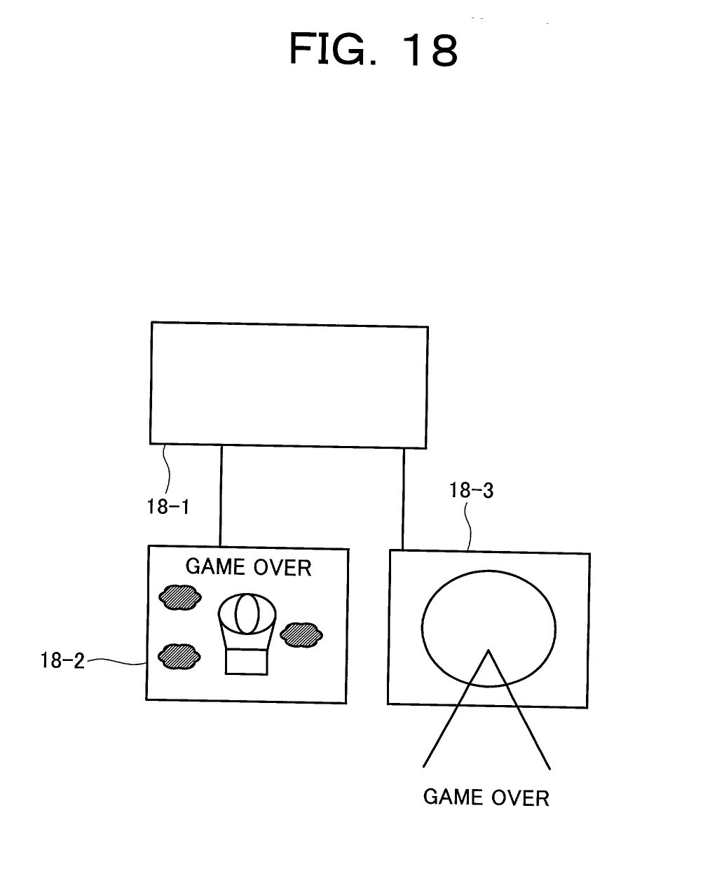

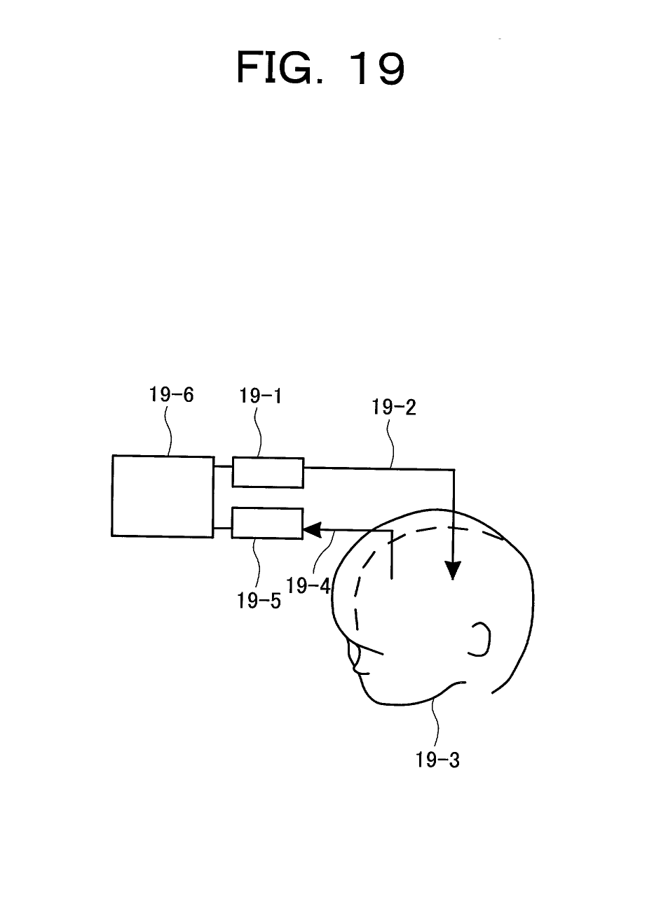

Next, the method of giving an instruction to the subject to stop the measurement (game) will be described referring to FIG. 18. 18-1 in the figure is an electronic calculator, and this electronic calculator comprises a display screen (18-2) and an audio announcement speaker (18-3) to realize an amusement system which reflects brain activity dependent on light intensity propagated in living tissue. To inform the subject that the measurement (game) has stopped as shown in FIG. 16, the message “game over” is first displayed on the display screen (18-2).

In other words, by displaying the message “game over” on the screen, the screen display is arranged to be different to the ordinary display during the game. In an identical way, a message to the effect that measurement has stopped, e.g.,“game over!”, is played from the audio announcement speaker (18-3). During an ordinary game, special effect sounds are played over the speaker to increase the sense of presence, but this message is arranged to be different from the sounds normally heard during the game.

As described above, the amusement system provided by this invention measures the concentration of a metabolite or its concentration variation in living tissue reflecting a mental state, and reflects this measurement result in an object displayed on a screen. As a result, the object on the screen can be controlled without using an input device such as a mouse, joystick or handle. This provides a new amusement system, and further permits enjoyment of an amusement system without using the hands or feet.

What is claimed is:

1. An amusement system using an optical instrumentation method for the living body comprising at least one light illumination device for irradiating the subject with light, at least one light detector which condenses the transmitted light irradiated by the light illumination device and propagated through the living body, a display unit provided with a screen which displays at least one object, and a computing part which controls the display unit based on a measurement signal related to the intensity of the transmitted light measured by said light detector, wherein a state comprising at least one of said objects displayed on said screen varies according to the intensity variation of the signal measured by said one or more light detectors.

2. An amusement system using a living body optical instrumentation method comprising at least one light illumination device for irradiating the subject with light, at least one light detector which condenses the transmitted light irradiated by the light illumination device and propagated through the living body, a computing part provided with a screen which displays at least one object, and a computing part which controls the display unit based on a measurement signal related to the intensity of the transmitted light measured by said light detector, wherein a state comprising at least one of said objects displayed on said screen varies according to the intensity variation of the signal measured by said one or more light detectors.

3. An amusement system using an optical instrumentation method for the living body comprising at least one light illumination device which irradiates plural living bodies with light via waveguides, at least one light detector which condenses the transmitted light irradiated by the light illumination device and propagated through the living body, a display unit provided with a screen which displays at least one object, and a computing part which controls the display unit based on a measurement signal related to the intensity of the transmitted light measured by the light detector, wherein a state comprising at least one of said objects displayed on said screen varies according to the intensity variation of the measured signal measured by said one or more light detectors.

4. An amusement system using an optical instrumentation method for the living body wherein the light illumination device, light detector, display unit and computing unit are built into the same information terminal, and parts of the light illumination device and light detector are connected to a probe of the information terminal.

5. An amusement system using an optical instrumentation method for the living body wherein the computing part has a function for storing the cumulative time and a reference illumination period for which light is irradiated by the light illumination device, and a speaker which emits a sound, and when the cumulative time during which light is irradiated by the light illumination device exceeds the reference illumination period, a command is issued to modify the arrangement on the display screen or a command is issued to modify the sound emitted by the speaker.

6. An amusement system using an optical instrumentation method for the living body wherein the computing part issues a command to modify the arrangement on the display screen or modify the sound emitted by the speaker when the intensity of the transmitted light exceeds a predetermined threshold value even if the cumulative time during which light is irradiated by the light illumination device is within said reference illumination period.
